(19) United States
(12) Patent Application Publication
(10) Pub. No.: US 2014/0255409 A1
(43) Pub. Date: Sep. 11, 2014

(54) METHOD FOR EXTRACORPOREAL ELIMINATION OF ONE OR MORE COMPONENTS FROM BLOOD

(71) Applicant: GLYCOREX TRANSPLANTATION AB, Lund (SE)

(72) Inventor: Kurt Nilsson, Lund (SE)

(21) Appl. No.: 14/354,834

(22) PCT Filed: Oct. 29, 2012
(86) PCT No.: PCT/SE2012/051173

§ 371 Date: Apr. 28, 2014

(30) Foreign Application Priority Data
Oct. 28, 2011 (SE) 1100803-4

(51) Int. Cl. B01D 15/38

(52) U.S. Cl. CPC B01D 15/3804; B01D 15/3809; B01D 15/3828
USPC 424/137.1; 210/691; 210/661; 210/656; 514/13.5

(57) ABSTRACT

A method for extracorporeal elimination of one or more components from blood where whole blood or blood plasma is added to a blood treatment device containing an adsorbent which binds the one or more components is disclosed. The adsorbent comprises at least one matrix to which at least one ligand having specific binding affinity to said one or more component is covalently bound. The ligand comprises a glycosidically bound aglycon and at least one saccharide, preferably blood group determinant A, blood group determinant B, blood group H determinant, a P antigen, or a Pk antigen; or said ligand is an amino acid, a peptide or an antibody. Methods for treatment of transplant recipients, and to use a blood treatment device is also disclosed.

### TECHNICAL FIELD OF THE INVENTION

The present invention refers to a method for extracorporeal elimination of one or more components from blood, as well as to methods for treatment of transplant recipients and to use of a blood treatment device.

### BACKGROUND ART

Blood treatment is daily performed in different embodiments on a large amount of patients or on donor blood. The purpose of the blood treatment is e.g. to treat different disease conditions or to separate or extract certain components from the blood.

A method called immunoadsorption, normally abbreviated IA, in which a biospecific column is used for an extracorporeal blood treatment, has been developed with a view to providing a more specific treatment or elimination of certain components in blood. An IA column contains a material consisting of a matrix having at least one ligand covalently bound thereto. Said ligand often consists of or comprises a peptide, a protein, an antibody or a carbohydrate structure. The ligand often contains an aglycon for the covalent binding of low molecular structures. The aglycon separates the active component in the ligand from the matrix. IA provides a specific treatment, i.e. a specific elimination or reduction of the target substance for the treatment, and e.g. can immunoglobulin G be separated from other proteins in blood, also from proteins having a similar molecular weight or size as immunoglobulin G. An example of an IA column is a protein A containing column. These columns contain protein A bound to a polymeric carrier material, e.g. cross-linked agarose. Other examples are immunoglobulin based columns for specific elimination of immunoglobulins or for separation of other proteins or components from blood plasma. Further examples are columns having a covalently bound blood group saccharide with a view to specifically binding that part of antibodies in blood which is specific for the blood group determents A and B, respectively, while other antibodies and blood components flow through the column.

In IA a plasma filter, like in PE (plasma exchange) or a centrifuge is used, which continuously separates the plasma from the blood cells. Then the blood plasma is transported via a tube to a column, in which the target protein or component is bound and thereby is specifically separated from other blood plasma components. Blood plasma which has passed the column is continuously transported back to the patient.

### SUMMARY OF THE INVENTION

The present invention refers to a method as defined in independent claim 1, more precisely to a method for extracorporeal elimination of one or more components from blood, wherein it comprises the following steps:
a) adding whole blood or blood plasma containing said one or more components to a blood treatment device containing an adsorbent, which comprises at least one matrix to which at least one ligand having specific binding affinity to said one or more components is covalently bound, wherein said one or more components are bound to said ligand, said ligand being covalently bound to the matrix and comprises a glycosidically bound aglycon and at least one saccharide, preferably blood group determinant A, blood group determinant B, blood group H determinant, a P antigen, or a Pk antigen, or said ligand is an amino acid, a peptide, or an antibody,
b) separation of the adsorbent having said one or more components bound thereto from the whole blood or blood plasma, and, optionally,
c) separation of said one or more components from the adsorbent, and, optionally,
d) analysis of said one or more substances.

Further embodiments of the present invention are disclosed in the subsequent dependent claims.

In a further aspect, the present invention also refers to methods for treatment of transplant recipients.

In still a further aspect, the present invention refers to use of a blood treatment device in the method according to the present invention.

### DETAILED DESCRIPTION OF DIFFERENT EMBODIMENTS OF THE INVENTION

The present invention involves products comprising at least one blood treatment container and at least one adsorbent consisting of or comprising at least one matrix and at least one ligand covalently bound thereto. The adsorbent is used for specific binding or reduction of antibodies, or proteins or other specific components in the inventive method for extracorporeal elimination of one or more components from blood. Examples of the blood treatment device involved in the present invention is e.g. a blood bag or a blood plasma bag. The adsorbent is present in said blood treatment device, which may contain blood, blood plasma, partially purified blood plasma, or partly purified blood components, e.g. immunoglobulin.

Throughout the application text, the expression “blood” is generally intended to mean whole blood, but here it also covers partially purified blood components, e.g. IVIG (intravenous IG), unless otherwise is stated. The expression “blood plasma” used throughout the application text is intended to also cover partly purified plasma, unless otherwise is stated. In general, the component to eliminate in the inventive method is originally present in whole blood, but may also be present in blood plasma separated from said whole blood. The expression “blood treatment device” used throughout the application text is intended to mean a device with which the method for the extracorporeal elimination of one or more components from blood is performed. An example of a blood treatment device is a blood bag, a blood plasma bag or a column. These devices are commercially available. The blood treatment device may contain the adsorbent before the blood or blood plasma to purify is addod, or vice versa.

In one embodiment the adsorbent, e.g an antibody or protein binding adsorbent, is added to a blood bag, a blood plasma bag or a column from a separate adsorbent container. The adsorbent container may be a tube, a plastic bag, a syringe or a container made of glass. In the case the adsorbent container is a plastic bag, the adsorbent may be transferred to the blood bag or blood plasma bag via a plastic tube connected to said bag. The transfer can take place before or after the addition of blood or blood plasma to the blood bag or blood plasma bag. The plastic bag with adsorbent intended for injection to a blood bag or a blood plasma bag may be sterilized e.g. via autoclaving. In the case the adsorbent is present in a tube, the adsorbent may be transferred via the plastic tube, or via a connection tube connected to the plastic tube, to the blood bag or the blood plasma bag. In the latter example, the plastic tube containing the adsorbent and the connection tube may have been end-sterilized, e.g. by autoclaving of the sealed plastic tube containing the adsorbent before use. As stated above, the adsorbent may also be present in a blood bag or blood plasma bag. Also in this case the blood bag or the blood plasma bag containing the adsorbent have been sealed and end-sterilized, e.g. by autoclaving. By rotation or shaking of the blood bag or blood plasma bag after the addition of the adsorbent, or by agitation of the adsorbent in the blood bag or blood plasma bag, the adsorbent is brought in contact with the blood or blood plasma. Thereby the antibody, protein or any other component in the blood or blood plasma is specifically bound to the adsorbent. E.g., partially purified immunoglobulin may be bound to the adsorbent in such a way. After the binding the adsorbent having the desired components bound thereto is separated from the blood or blood plasma. Thereby, the intended components are separated from the blood, which is the purpose in one aspect of the present invention.

Optionally, as a final step, the antibody, protein or other component bound to the adsorbent is eluated from the adsorbent by e.g. changing the pH of the adsorbent. This may be accomplished by e.g. adding a buffer having a lower pH, e.g. a glycine buffer having a pH which is lower than neutral. Thereby, the specific binding between the adsorbent and the component bound thereto is broken. In such a way the adsorbent may optionally be reused after said separation.

In an alternative embodiment, the adsorbent may also be present in a blood bag or blood plasma bag before the blood or blood plasma is added. In such a way, the addition of the adsorbent may be accomplished by injection of the adsorbent into the blood bag or blood plasma bag. Such an injection of the adsorbent may be accomplished by the use of a tube connection between the blood bag or blood plasma bag and the adsorbent container. In such a way, the adsorbent is injected from an adsorbent container containing a desired amount of adsorbent, an amount corresponding to the desired amount in the blood bag or blood plasma bag, or a larger amount of adsorbent with a view to making possible injection to several blood bags or blood plasma bags from an adsorbent container.

The adsorbent present in the blood treatment device contains at least one covalently bound ligand, as stated above. Each ligand comprises at least one saccharide and a glycosidically bound aglycon in the reducing end of the saccharide. The aglycon is present with a view to covalently binding the saccharide to the matrix and with a view to sterically separating the saccharide from the matrix. Non-limiting examples of the saccharide are blood group determinant A, blood group determinant B, P antigen, K antigen, and other blood group determinants. Other non-limiting examples of the ligand are an amino acid, a peptide, an antibody, or a protein.

The blood group A and B determinants may be present in the form of a blood group A-trisaccharide determinant and a blood group B-trisaccharide determinant, respectively, and/or may e.g. be present in the form of one of, or a combination of two or more of, a blood group A determinant of subtype 1, 2, 3 or 4, as well as a blood group B and H determinant, respectively.

An example of the blood group A-trisaccharide determinant is GalNAcα1-3(Fucα1-2)Galβ1-.

Examples of blood group A determinant subtypes 1, 2, 3, and 4, respectively, are:
GalNAcα1-3(Fucα1-2)Galβ1-3GlcNAcβ1-,
GalNAcα1-3(Fucα1-2)Galβ1-3GlcNAcβ1-3Galβ1-4Glcβ1-,
GalNAcα1-3(Fucα1-2)Galβ1-4GlcNAcβ1-,
GalNAcα1-3(Fucα1-2)Galβ1-4GlcNAcβ1-3Galβ1-4Glcβ1-,
GalNAcα1-3(Fucα1-2)Galβ1-3GalNAcβ1-,
GalNAcα1-3(Fucα1-2)Galβ1-3GalNAcβ1-3Galβ1-4Glcβ1-,
GalNAcα1-3(Fucα1-2)Galβ1-3GalNAcα1-,
GalNAcα1-3(Fucα1-2)Galβ1-3GalNAcα1-3Galβ1-4Glcβ1-.

An example of the blood group B-trisaccharide determinant is:
Galα1-3(Fucα1-2)Galβ1-.

Examples of blood group B determinant subtypes 1, 2, 3, and 4 are, respectively:
Galα1-3(Fucα1-2)Galβ1-3GlcNAcβ1-,
Galα1-3(Fucα1-2)Galβ1-3GlcNAcβ1-3Galβ1-4Glcβ1-,
Galα1-3(Fucα1-2)Galβ1-4GlcNAcβ1-,
Galα1-3(Fucα1-2)Galβ1-4GlcNAcβ1-3Galβ1-4Glcβ1-,
Galα1-3(Fucα1-2)Galβ1-3GalNAcβ1-,
Galα1-3(Fucα1-2)Galβ1-3GalNAcβ1-3Galβ1-4Glcβ1-,
Galα1-3(Fucα1-2)Galβ1-3GalNAcα1-,
Galα1-3(Fucα1-2)Galβ1-3GalNAcα1-3Galβ1-4Glcβ1-.

Optionally, the adsorbent contains a ligand containing the blood group A-trisaccharide determinant GalNAcα1-3(Fucα1-2)Galβ1- together with one or more of the blood group A subtypes above, and/or together with the blood group B-trisaccharide determinant Galα1-3(Fucα1-2)Galβ1- and, optionally one or more of the blood group B-trisaccharide determinant subtypes listed above.

One example of an adsorbent used in the method according to the present invention is a saccharide bound to a matrix via an aglycon, wherein the aglycon is a monomer, a dimer, an oligomer or polymer.

Examples of the matrix are agarose, dextran, starch or starch derivatives, amylose, amylopectin, polyacrylamide, or any other polymer. In one embodiment cross-linked agarose is used as matrix. Cross-linked agarose is not soluble in blood or blood plasma and is therefore used in the form of porous gel beads.

As a non-limiting example of monomeric aglycon wherein the aglycon is glycosidically bound via —O— to the carbohydrate according to the example above, can be mentioned a monomeric aglycon which contains one or more of the structures —OPhNH—, —OEtPhNH—, or —O(CH₂)ₙ—NH—, wherein the NH group is bound directly to the matrix or is bound to the matrix via another chemical structure, which is one of the monomeric structures mentioned below. Alternatively, the structure can be bound to di-, tri- or oligomeric structures or to an amino acid, a peptide or a protein.

As another example, a carbohydrate with the above-mentioned type of glycosidically bound aglycon is optionally bound to a matrix via any one of the exemplified structures' amino group and another chemical structure, an example of such a structure bound to the matrix being a —C(O)—(CH₂)ₙ—O—CH₂— matrix or —(CH₂)ₙ—O—CH₂-matrix, wherein n is a whole number, preferably 1, 2, 3, 4, 5, 6, or higher, in order to create a further space between the carbo-hydrate ligand and the matrix. Thus, examples of adsorbents constructed according to these examples are: carbohydrate-OEtPhNH—C(O)—(CH₂)ₙ—O—CH₂-matrix and carbohydrate-OEtPhNH—C(O)—(CH₂)ₙ—NH—CH₂-matrix. Several other methods for the coupling of the ligand to the matrix exist.

The choice of the structure of the aglycon is made by the expert in the field. The aglycon can also contain an amino acid, a peptide or a protein.

One or more saccharide structures can be used bound to a matrix, such as polymer particles. The choice of the ligand, e.g. the carbohydrate or the carbohydrate derivative, the concentration of the ligand on the matrix, the method for covalent binding of the ligand to the matrix, the conditions for covalent binding of the ligand to the matrix (for example temperature, pH, concentration of ligand and matrix, the washing of the matrix after covalent coupling of the ligand), is made by the expert in the field for the specific application.

As stated above, the ligand may also be a protein or a peptide which may be bound to the antibody or protein which is desired to reduce in blood or blood plasma, or in completely or partially purified blood plasma.

Examples of a preferred matrix is an agarose based matrix, for example agarose or cross-linked agarose which normally is porous or macro-porous, thus containing pores which allow entrance of proteins or antibodies into said pores when a large amount of ligand is bound, cellulose, cross-linked cellulose, and a plastic filter, used in e.g. hemofiltration, dialysis, or plasma exchange.

In one embodiment cross-linked agarose having a lower agarose content is used, e.g. 2-3% on dry weight basis, with a view to allowing quicker access to the pores within the matrix for the binding of larger plasma components, e.g. antibodies of the type IgG and/or IgM. Examples of specific antibodies are anti-A, anti-B, anti-GM1, and anti-Galα1-4Galβ1-4Glc antibodies.

An example of such a commercially accessible agarose is Sepharose 2B or CL 2B, wherein CL is a cross-linked variant of agarose, but also agarose having a higher dry substance content may be used, e.g. the commercially available Sepharose 4B or CL 4B, CL 6B or Sepharose 4FF (Fast Flow). Also other similar types of agarose may be used.

The linkage between the carbohydrate containing ligand and the matrix is preferably covalently stable, and examples thereof are an amide (NH—CO), N—C, or O—C linkage. The former linkage is preferably formed by reacting an activated carboxyl group (activated with carbodiimide and or N-hydroxy-succinimide) on the ligand, or the matrix, with an amino group on the matrix, or the ligand.

The blood treatment device is e.g. of interest for purification of intravenous immunoglobulin fractions from anti-A, and/or anti-B and/or anti-H antibodies, for removal of said antibodies from human plasma or human blood in connection with transfusion of blood or of blood plasma, or for removal of anti-A and/or anti-B antibodies from partially purified immuno-globulin fractions in the preparation of intravenous immunoglobulin or for purification of other plasma components, and for preparation of human plasma or freeze-dried human plasma with reduced anti-A and/or anti-B, and/or anti-H antibody content.

For example, by treating human blood or human blood plasma of blood group 0 with the method according to the invention, the content of anti-A and anti-B antibodies can be reduced in human blood or human blood plasma of blood group 0. This facilitates use of blood group 0 donor blood or blood plasma, or blood group 0 donor platelets, for transfusion to patients having other blood groups than blood group 0. Also, cells and/or proteins from blood group 0 donors can be used for donation to patients having other blood groups.

As another example, it can be mentioned that by the treatment of human blood, or of human blood plasma of blood group A with the method according to the invention, the content of anti-B antibodies can be reduced in human blood or human blood plasma of blood group A. This facilitates use of blood group A donor blood or blood plasma for transfusion to patients of other blood groups.

The adsorbent, containing matrix and covalently bound ligand, may be used in beaded form as a gel suspension, i.e. gel beads suspendend in aqeuous liquid or buffer. The exact liquid or buffer is chosen by the expert in the field. As an example can be mentioned sterile saline buffer used for injection in patients.

In one embodiment of the invention, for the treatment of human blood or blood plasma of blood group 0, a gel suspension containing gel beads with covalently bound blood group A-ligand(s) and blood group B ligand(s) exemplified above, is used. The blood group A-ligand consists of a blood group A-trisaccharide ligand and at least one of blood group A-tetrasaccharide of subtype 1, 2, 3 and or 4 as described above. In this example, the ratio of the contents (for example mg saccharide/mg of settled gel bead) of blood group A-trisaccharide to the content of each one of the tetrasaccharide subtypes, for example a value of ¼, ½, 1/1, 2/1 or 4/1, or the ratio has a value between these values. Each A-ligand can for example be coupled simultaneously to the matrix, forming the gel beads, or be coupled separately to separate gel beads. The type combinations as exemplified above for A-ligand containing gel beads can as an example be applied for the choice of B-ligand containing gel beads (for example the ratio of B-trisaccharide to B-tetrasaccharide subtypes and coupling). The ratio of A- to B-ligand can be for example be 1/3, 1/1 or 3/1, or be a value between these values. The exact choice of the above parameters is made by the expert in the field.

The total quantity of ligand can be chosen to be e.g. 0.1, 0.5, 1.0, 2.0, 4.0 mg per ml settled gel beads or a value between these values.

The concentration of ligand containing gel beads in the gel suspension (ratio of settled volume of gel beads to the total volume of the gel suspension) is chosen by the expert in the field. The volume of settled gel beads is determined by the application and is also chosen by the expert in the field. As an example can be mentioned that 1, 2, 3, 4 or 5 ml settled gel beads with covalently bound A- and B-ligand is used for reduction of anti-blood group A and anti-blood group B antibodies in human blood or human blood plasma in a blood bag containing 200, 300, 500 or 750 ml, or a value between these values, of human blood or human blood plasma.

When an adsorbent in the form of gel beads is used, the bead size of the matrix is chosen by the expert in the field. For example, smaller gel beads can allow for a more rapid diffusion into the pores of the gel beads and thus a more rapid binding reaction of the antibody or of the protein. For example, gel beads in the size range 20 to 200 μm, or gel beads with a more narrow range within that range, can be chosen.

In one embodiment the adsorbent may be injected at sterile conditions via a sterile tube from a sterilized adsorbent container, which is an example of a blood treatment device used in the method according to the present invention. The adsorbent may e.g. be injected in a blood bag or blood plasma bag, as mentioned above. In the latter example, the adsorbent may e.g. be present in a syringe, in a container made of glass, in a plastic tube, or in a plastic bag from which the gel suspension may be injected into the blood bag or the blood plasma bag. In the case when the absorbent is present in a plastic bag, an adsorbent may be transferred from the plastic bag via e.g. a plastic tube which is connected to the blood bag or blood plasma bag in a sterile way. Thereafter, the treatment of the blood or the blood plasma takes place.

Thus, the adsorbent present in the plastic bag or a plastic tube for injection to a blood bag or blood plasma bag is one example of a blood treatment device involved in the method according to the present invention. This device is used for maintenance and transport of the adsorbent, as well as for storage before injection of the adsorbent to a blood bag or a blood plasma bag. This device may have been produced during validated certified conditions and may have been end-sterilized by e.g. autoclaving.

Today several bags that are used as containers for injection solutions exist. This type of bag is a non-limiting example of a bag which can be used according to the invention. According to the invention a plastic bag which is biocompatible with blood and blood plasma and which allows end-sterilization is preferably used. As the bag material e.g. PVC, or a polyolefin, polypropylene, may be used. The bag has a connection, e.g. via a tube extending into the bag which on one hand allows loading of a desired amount of gel suspension in the bag, and on the other hand allows sealing via the tube for end-sterilization and for sterile connection (e.g. by means of welding of the tube from the bag to the tube of the blood bag or the blood plasma bag by means of a sterile connecting device) to the blood plasma bag or to the blood bag (with a view to being able to transfer the gel suspension to the blood bag or the blood plasma bag after the connection but before use). E.g., in a preferred embodiment the inner volume of the bag holds the desired volume of the gel suspension (exemplified above) and the bag has such a strength that the gel suspension in the bag allows end-sterilization, e.g. by means of steam sterilization or autoclaving, and, in addition, the bag has a permeability for liquid (alone or together with an outer storage bag) which is low enough during storage before use. The tube used for the connection may consist of one or more pieces, e.g. one or two interconnected tubes depending on the need. The plastic material (PVC, polyolefin) and the dimensions (length, diameter) normally used for tubes also apply for examples of useful tubes here. This does not limit the scope of the invention. These and other parameters (e.g. the parameters chosen for the end-sterilization, the length and the inner dimension of the connecting yube, and the connection during sterilization and storage, and the connection of the blood bag before the transfer of the gel suspension to the blood bag) are chosen by the skilled person in the art and does not limit the scope of the invention.

Optionally, as mentioned above, other embodiments than a bag, e.g. a tube, for the storage of the gel suspension before the use may be chosen and be used in the same way as mentioned above, e.g. in the form of a tube. In this example the gel suspension is loaded within a tube (consisting of e.g. at least one material, e.g. PVC or a polyolefin, or is comprised of a combination of at least two tubes). This tube is sealed in both ends, sterilized, stored, and connected to the blood bag or blood plasma bag, followed by transfer of the gel suspension to the blood bag. For e.g. a 5 ml gel suspension, as a non-limiting example, a tube having an inner diameter of 4 mm may be used, as well as a length between the sealings of at least 40 cm.

As a further non-limiting example of the product according to the invention, a product characterized by a gel suspension in a blood bag or blood plasma bag may be mentioned.

E.g., the adsorbent may be made of cross-linked agarose having a covalently bound saccharide containing ligand, as mentioned above. The adsorbent may e.g. be suspended in a saline buffer or a PA buffer or any other aqueous solution selected by the expert in the field. The blood or blood plasma bag is made of a blood compatible material which also is autoclavable, e.g. PVC or polyolefine, or any other plastic material. Said bag material, as well as the volume of the adsorbent (ml settled volume), the suspension volume, the plastic bag volume, the plastic connection to the plastic bag, the plastic composition of the connection (e.g. PVS or polyolefine), and the connection to the blood bag are chosen by the person skilled in the art. Anyone of the adsorbent volumes presented above in the present application may be used for treatment of e.g. 200 ml blood or blood plasma. The volume of the plastic bag is chosen dependent on the volume of the adsorbent. Examples of the volume are 2, 3, 4, 6, 10, or 20 ml adsorbent suspension, or any volume therebetween, in a plastic bag having an inner volume of between 25 and 40 ml. These parameters are chosen by the person skilled in the art. Optionally, the plastic bag containing the adsorbent has been produced at validated and certified conditions, as well as the introduction of an adsorbent in a plastic bag and sealing of the tube connetion. Optionally, this blood treatment device has been end-sterilized, e.g. by autoclaving.

Blood bags and blood plasma bags for clinical use and handling of donor blood/donor blood plasma are commercially available, and these are examples of blood bags and blood plasma bags which are useful according to the invention. In the method according to the invention blood or blood plasma is transferred to the blood bag or the blood plasma bag as a next step. As a non-limiting example according to the invention, this transfer may be made from a blood donor to a blood bag containing the gel suspension, as disclosed above. In this example the blood bag may constitute the primary bag in a system of interconnected blood bags which continuously are used for the collection of blood from blood donors and from which blood, blood plasma, blood platelets, and blood components, such as IVIG, respectively, are obtained. In another embodiment the gel suspension may be present in a blood plasma bag, to which blood plasma obtained is transferred from another blood plasma bag. In both of the exemplified cases the bag is thereafter rotated or is mixed in another way with a view to achieving suspension and blending of gel beads in the blood or blood plasma, and to facilitating binding of e.g. anti-A and/or anti-B antibodies to the gel suspension. The rotation speed, contact time, and temperature is chosen by the skilled person in the art and does not limit the scope of the invention. As a non-limiting example, 0.5, 1, or 2 revolutions per second, or a value between these values, and a time of 10, 30, 60, or 120 min, or a value between these values, may be mentioned.

As a last step according to the invention, the gel suspension is separated from the blood or the blood plasma. This can be done by means of e.g. filtration or centrifugation and does not limit the scope of the invention.

The present invention also refers to the product and the use thereof obtained with the product and the method according to the invention, i.e. blood, blood plasma, and antibodies having a reduced level of e.g. anti-A and/or anti-B antibodies.

After the binding of the antibody, protein or other component to the adsorbent, e.g. anti-A or anti-B antibodies in the blood or blood plasma, the adsorbent is separated from the blood or blood plasma, or from the completely or partially purified blood, in the latter case e.g. from completely or partially purified IgG or IVIG.

The separation of the adsorbent from the blood or blood plasma is e.g. made by use of a filter connected to the blood bag or blood plasma bag, preferably via a tube connected to the blood bag or the blood plasma bag. The blood or blood plasma is allowed to pass through said filter, but not the adsorbent. The separation of the adsorbent may alternatively be performed by use of centrifugation. The flow through the filter, the pore size of the filter and the design of the filter, as well as the centrifugation conditions, is chosen by the person skilled in the art. Several commercially available filters exist.

The amount of adsorbent per volume of blood or blood plasma is chosen by the person skilled in the art. E.g., 0.1 ml, 1 ml, 2 ml, 3 ml, 4 ml, 5 ml or more, or a volume therebetween, of the product may be used per 100 ml blood or blood plasma. In the case of larger volumes of blood or blood plasma, the contact time, the temperature, the rotations per minute of the blood bag and other parameters in connection with the binding of the antibody protein or other components to the adsorbent are chosen by the person skilled in the art.

The adsorbent may also be used in a column, in which blood or blood plasma, or partially purified blood, is brought to pass via a tube from the blood bag or blood plasma bag to the column and via an another tube out from the column, e.g. to a second blood bag or blood plasma bag. E.g., the column may be connected after the blood bag or blood plasma bag, and blood or plasma is allowed to pass through the column. The size of the column may vary dependent on the application. At a size of 30 ml, 60 ml, 100 ml, 300 ml, or a volume therebetween or more, 2, 4, 6, 10, 20, or an amount therebetween, blood bags (normally having a volume of 200 ml, 300 ml, 400 ml, 700 ml or a volume therebetween, or higher) be connected in parallell to the column, and the passage from the bags can take place in parallell with a common flow of blood or blood plasma through the column. Alternatively, passage of blood plasma from each bag may take place in sequence through the column, and each bag may be connected one by one to the column, wherein blood plasma from each bag may pass at the same time or in sequence. Several bags may be connected to the column via a coupling device having several tube connections, e.g. one from each bag, and plasma may be allowed to pass through the column in sequence or at the same time from the connected bags via open/shut taps on the coupling device. In such a way, e.g. 5, 10, 20 or more bags may be connected to the column. Collection of blood plasma which has passed the column may be made to several bags at the same time or to only one bigger bag. When a smaller column or when a smaller amount of suspended product in a blood bag as disclosed above is used, the treated plasma may be transported to the patient via a tube connected after the column or the filter. The column is preferably fitted with an inlet and an outlet for the plasma, as well as a filter with a view to avoiding contamination of passed plasma with matrix. The matrix particle is constructed and chosen in such a way that it resists the flow pressure.

Normally, the amount of adsorbent is adapted to the blood or blood plasma volumes to be treated. In the case of a blood treatment device containing pooled blood or blood plasma from several different blood donors or blood donor occasions, the blood or blood plasma volume to be treated with this product may e.g. be in the dimension 10 L, 100 L, 200 L, or volumes therebetween, or higher or lower volumes. The amount of the product used is adapted by the expert in the field.

The passage through the column may also take place by use of a so-called fluidized bed process, in which the flow through the column runs from the lower part of the column to an outflow from the upper part of the column. By adapting the amount of adsorbent and the flow of blood, blood plasma or completely or partially purified blood plasma, the adsorbent, e.g. in the form of non-soluble gel beads, may be maintained as fluidized in the flow. This embodiment is preferable in applications in which higher flows are desired or in which e.g. whole blood is to be treated with the adsorbent.

The above mentioned products can also be used for binding of anti-A and/or anti-B and/or anti-H antibodies from human blood immunoglobulin fractions, in either purification stage, initial, intermediate or finished product (intravenous immunoglobulin). The specific volume of the blood treatment device and the chosen variant of the blood treatment device as described above, is chosen by the expert in the field and do not limit the scope of the invention. This depends on, e.g. the desired exact application and the quantity of antibodies to be removed. The blood treatment device can be used in large scale application for production of e.g. human plasma or intravenous immunoglobulins with reduced or minimal content of mentioned blood group specific antibodies.

If the blood treatment device is used in the form of a matrix which forms a beaded gel suspension with the fluid to be treated, e.g. containing beaded cross-linked agarose as matrix with covalently bound ligand, the blood treatment device can be provided with either one of for example donor blood, donor blood plasma, fractionated blood proteins, such as immunoglobulin fractions, and the blood treatment device separated from said donor blood blood, donor plasma or immunoglobulin fractions after binding of anti-A and/or anti-B and/or anti-H antibodies. The bead size and other treatment parameters are chosen by the expert and do not limit the scope of the invention. For example, one of the bead size ranges of the beaded product mentioned above can be chosen depending on the exact application.

The quantity of ligand in the blood treatment device is typically chosen to contain 0.1, 0.5, 1.0, 2.0, 10, or 20 mg ligand per mL volume of adsorbent or any value between these values. The value is chosen by the expert in the field.

In one embodiment of the invention at least the final stages of the production of the blood treatment device is performed in clean rooms, and preferably the reagents and clean room(s) used are certified according to international standards and or requirements for the product application. In another specific embodiment of the invention the blood treatment device is end-sterilised with steam and/or autoclaved to ensure a sterile blood treatment device before use.

In another embodiment of the invention, the to the blood treatment device bound anti-A and/or anti-B and/or anti-B antibodies, toxin, bacteria, and virus, can be eluted from the blood treatment device, analyzed and used for various applications, such as purification or analyses.

When the adsorbent involved in the invention is in a column as described above, the column can be used for an extracorporeal treatment of a recipient of an ABO-incompatible graft. In transplantation of blood group A1 donor organs or cells to A2 recipients, there is a risk that the A2 recipient contains antibodies specific towards certain variants of blood group A structures. By treating the A2 recipient with the variant of product containing the blood group A variant, these antibodies can be reduced or eliminated thus reducing the risk of side effects due to those antibodies after transplantation. More broadly, certain blood group 0 and certain blood group B patients contain antibodies not only towards A-trisaccharides, but also towards longer A-structures and these patients can be treated with the blood treatment device involved in the invention with a view to removing said antibodies and facilitating blood group incompatible transplantation from A donors. In one embodiment of the invention, this can be performed especially when there is a relatively low titer reduction with conventional A trisaccharide products on the market or there is a persistent rebound of antibodies.

Similarly, certain patients contain antibodies also towards H-structures (Bombay type), and in treatment with the blood treatment device containing these structures according to the invention these antibodies can be removed and the patient can be transplanted.

In addition, in blood group incompatible stem cell transplantation, the product can be applied to treat patients with high anti-A, and/or anti-B and/or anti-H titers with a view to avoiding problems with anti-A, anti-B and anti-H specific antibodies.

In one more specific embodiment of the invention the blood treatment device above containing only the above described ligands, without the above described matrix, is used for inhibition of said antibodies, e.g. during birth or blood group incompatible transplantation or for inhibition in vivo, for use against certain blood group A receptor and other carbohydrate dependent infectious deseases, e.g. soluble variants of the polymer based blood treatment device having covalently bound blood group A determinant, ganglioside structures, other sialylated structures, or Galα1-4Galβ1-4Glc for binding or inhibition of bacteria, virus, and toxins, e.g. via oral administration, or inhibition of urinary tract bacteria by use of other adsorbents adapted thereto.

1. A method for extracorporeal elimination of one or more components from blood, wherein it comprises the following steps:
a) adding whole blood or blood plasma containing said one or more components to a blood treatment device containing an adsorbent, which comprises at least one matrix to which at least one ligand having specific binding affinity to said one or more components is covalently bound, wherein said one or more components are bound to said ligand, said ligand being covalently bound to the matrix and comprises a glycosidically bound aglycon and at least one saccharide, preferably blood group determinant A, blood group determinant B, blood group H determinant, a P antigen, or a Pk antigen, or said ligand is an amino acid, a peptide, or an antibody,
b) separation of the adsorbent having said one or more components bound thereto from the whole blood or blood plasma, and, optionally,
c) separation of said one or more components from the adsorbent, and, optionally,
d) analysis of said one or more substances.

2. The method according to claim 1, wherein the blood treatment device is a whole blood bag, a blood plasma bag or a column.

3. The method according to claim 1, wherein the adsorbent contains at least one ligand containing a blood group A-trisaccharide determinant chosen from GalNaca1-3 (Fucα1-2)Galβ1-, and subtype 1, 2, 3, or 4 thereof, preferably
GalNAcα1-3(Fucα1-2)Galβ1-3GlcNAcβ1-,
GalNAcα1-3(Fucα1-2)Galβ1-3GlcNAcβ1-3Galβ1-4Gicβ1-,
GalNAcα1-3(Fucα1-2)Galβ1-4GlcNAcβ1-,
GalNAcα1-3(Fucα1-2)Galβ1-4GlcNAcβ1-3Galβ1-4Gicβ1-,
GalNAcα1-3(Fucα1-2)Galβ1-3GalNAcβ1-,
GalNAcα1-3(Fucα1-2)Galβ1-3GalNAcβ1-3Galβ1-4Gicβ1-,
GalNAcα1-3(Fucα1-2)Galβ1-3GalNAcα1-,
GalNAcα1-3(Fucα1-2)Galβ1-3GalNAcα1-3Galβ1-4Gicβ1-.
and/or at least one ligand containing a blood group 8-trisaccharide determinant chosen from Galα1-3(Fucα1-2)Gall31-, and subtype 1, 2, 3, or 4 thereof, preferably
Galα1-3(Fucα1-2)Galβ1-3GlcNAcβ1-,
Galα1-3(Fucα1-2)Galβ1-3GlcNAcβ1-3Gaiβ1-4Gicβ1-,
Galα1-3(Fucα1-2)Galβ1-4GlcNAcβ1-,
Galα1-3(Fucα1-2)Galβ1-4GlcNAcβ1-3Gaiβ1-4Gicβ1-,
Galα1-3(Fucα1-2)Galβ1-3GalNAcβ1-,
Galα1-3(Fucα1-2)Galβ1-3GalNAcβ1-3Galβ1-4Glcβ1-,
Galα1-3(Fucα1-2)Galβ1-3GalNAcα1-,
Galα1-3(Fucα1-2)Galβ1-3GalNAcα1-3Galβ1-4Glcβ1-, and/or a H determinant.

4. The method according to claim 1, wherein said aglycon is a mono-, di-, tri-, tetra- or oligomeric aglycon.

5. The method according to claim 1, wherein the matrix is agarose or cross-linked agarose, preferably Sepharose 2B or CL, 4B or CLB8, CL 6B, or 4FF, cellulose or cross-linked cellulose, starch or derivatives thereof, amylase, amylopectin, or polyacrylamide, wherein the matrix preferably is present in beaded form as a gel suspension.

6. The method according to claim 1, wherein said one or more components to be eliminated from the blood are antibodies, preferably anti-A, anti-B, and/or anti-H antibodies, and/or proteins, toxins, bacteria, and viruses having binding affinity to said ligand.

7. The method according to claim 1, wherein it is used for purification of intravenous immunoglobulin fractions from anti-A, and/or anti-8 and/or anti-H antibodies, for removal of said antibodies from human plasma or human blood in connection with transfusion of blood or of blood plasma, or for removal of anti-A and/or anti-B antibodies from partially purified immunoglobulin fractions in the preparation of intravenous immunoglobulin or for purification of other plasma components, and for preparation of human plasma or freeze-dried human plasma with reduced 5 anti-A and/or anti-8, and/or anti-H antibody content.

8. The method according to claim 1, wherein the separation in step b) is performed by using a filter or by centrifugation.

9. The method according to claim 1, wherein in the case the blood treatment device is a column, blood or blood plasma from one or more blood bags or blood plasma bags are added to the upper part of the column in sequence or at the same time, wherein blood or blood plasma which has passed the column is emitted from the lower part of the column and is collected in one or more blood bags or blood plasma bags, wherein said column also contains a filter.

10. The method according to claim 9, wherein the column is a fluidized bed, wherein the blood or blood plasma is added to the lower part of the column and is emitted from the upper part of the column.

11. The method according to claim 9, wherein the matrix is made of beads having a size of 20-200 μm in the case blood plasma is added to the column and a size of 100-300 μm in the case blood is added to the column.

12. The method according to claim 1, wherein it alternatively comprises the following steps:
a) adding blood or blood plasma to a least one blood bag, blood plasma bag, or column,
b) providing the adsorbent in one or more adsorbent containers, connecting said at least one blood bag, blood plasma bag or column with said one or more adsorbent containers, preferably via a tube device,
c) adding the adsorbent to the blood or blood plasma, and
d) mixing the adsorbent and the blood or blood plasma in said at least one blood bag, blood plasma bag or column, preferably by agitation, rotation or shaking.

13. The method according to claim 12, wherein the adsorbent alternatively is added to said at least one blood bag, blood plasma bag or column before the blood or blood plasma is added.

14. The method according to claim 12, wherein said one or more adsorbent containers and/or the blood treatment device is end-sterilized or autoclaved.

15. The method according to claim 12, wherein the adsorbent container is a plastic bag, a syringe or a container made of glass.

16. A method for extracorporeal treatment of blood or blood plasma of a recipient of an ABO-incompatible graft or transplant, wherein the method according to claim 1 is used, and wherein the ligand on the adsorbent contains blood group A determinants.

17. A method for extracorporeal treatment of blood or blood plasma of blood group A2 recipients in connection with transplantation of blood group A1 donor organs or cells, wherein the method according to claim 1 is used.

18. A method for extracorporeal treatment of blood or blood plasma of blood group 0 and blood group B patients in connection with transplantation of organs or cells from blood group A donors, wherein the method according to claim 1 is used.

19. A method for prophylaxis and/or treatment of patients in connection with births, blood group incompatible transplantation, blood group A receptor and other carbohydrate dependent infectious diseases, preferably urinary tract infection, wherein a ligand as defined in claim 1 is orally administered to the patient.

20. A method of using a blood treatment device in a method according to claim 16.
